(19) United States
(12) Patent Application Publication
(10) Pub. No.: US 2015/0209093 A1
(43) Pub. Date: Jul. 30, 2015

(54) IMPLANTABLE MEDICAL DEVICE FOR RESTORING ALIGNMENT AND STABILIZING BONE FRACTURES AND METHODS OF USING THE SAME

(71) Applicant: James Dallis, Waxhaw, NC (US)

(72) Inventor: James Dallis, Waxhaw, NC (US)

(21) Appl. No.: 14/605,398

(22) Filed: Jan. 26, 2015

Related U.S. Application Data
(60) Provisional application No. 61/931,853, filed on Jan. 27, 2014.

(51) Int. Cl. A61B 17/82; A61B 17/80

(52) U.S. Cl. CPC A61B 17/82; A61B 17/8023

(57) ABSTRACT

There is disclosed an implantable medical device for restoring bone alignment and stabilizing a fracture, comprising a screwless bone plating system for contacting at least a portion of a bone. In one embodiment, the screwless bone plating system comprises at least one clamp comprising: a top portion; a bottom portion, and an attachment for securing the clamp to a bone without drilling a screw into the bone. Unlike current bone plating systems, the inventive clamp exhibits functionality in that it realigns a fracture when applied to the bone, and remains implanted in the body to stabilize the fracture. The medical device described herein may further comprise at two clamps for attaching to a bone, such as on opposite sides of a bone fracture and at least one bone plate that spans the fracture when attached to the two clamps. There is also disclosed a method of reducing and stabilizing a bone fracture, the method comprising contacting a fractured bone with an implantable medical device as described herein.

This application claims priority to U.S. Provisional Application No. 61/931,853, filed on Jan. 27, 2014, which is incorporated herein by reference in its entirety.

The present disclosure relates to implantable medical devices and methods for treating bone fractures. The implantable medical device comprises a screwless bone plating systems, including clamps, with plates optionally attached thereto, that can be quickly fastened and unfastened for easy repositioning during fracture fixation. The present disclosure also describes methods of reducing and stabilizing bone fractures using the described implantable medical device.

A bone fracture, which is defined as a break in the continuity of the bone, is typically caused by a high force impact or stress. Other common causes of bone fractures are pathological fractures that weaken bones, such as osteoporosis, bone or other cancers, and brittle bone disease, i.e., osteogenesis imperfecta.

Regardless of the cause of bone fracture, the various methods for treating such fractures involve the stabilization of the bone fragments. Once the fractured bone pieces are restored to their natural position, common techniques are used to immobilize the fractured bone. For example, bone plates, which are held into place using surgical nails, screws, and wires, are typically implanted to stabilize and “set” the fracture. The set fracture is then held into place by casting using plaster or fiberglass casts.

However, there are several drawbacks with current methods of treating bone fractures. From the patient's perspective, casts are described as heavy, hot, and awkward. From the surgeon's perspective, the use of typical nails, screws, plates, and wires are often hard to apply, making it difficult to fix the bone in correct alignment. Also, common plating techniques are not well suited for treating multiple fracture portions, and thus could require multiple plates.

Thus, there is a need for medical devices and related methods that can overcome at least some of the foregoing problems, while both simplifying and improving fracture repair. To solve at least some of these problems, the present Inventor has discovered a unique implantable medical device that greatly simplifies the process of rejoining and realigning the ends of broken bones by allowing the bone clamp, whether used alone or in combination with one or more bone plates, to have functionality in both reducing and stabilizing a fracture.

### SUMMARY

There is disclosed an implantable medical device for restoring bone alignment and stabilizing a fracture, comprising a screwless bone plating system for contacting at least a portion of a bone. In one embodiment, the screwless bone plating system comprises at least one clamp comprising: a top portion; a bottom portion, and an attachment for securing the clamp to a bone without drilling a screw into the bone, wherein the at least one clamp is capable of realigning a fracture when applied to the bone, and remains implanted in the body to stabilize the fracture.

In another embodiment, the implantable medical device described herein comprising a screwless bone plating system for contacting at least a portion of a bone. The screwless bone plating system comprises at two clamps for attaching to a bone, the clamps being located on opposite sides of a bone fracture and each comprising: a top portion comprising at least one hole or channel extending there-through, the hole or channel being adapted to receive a screw or connection post for attaching a bone plate to the clamp; and a bottom portion comprising an attachment for securing the clamp to a bone without drilling a screw into the bone. In this embodiment, the at least one bone plate is attached to the two clamps and span the bone fracture. Like the previous embodiment, the two clamps are capable of realigning the fracture when applied to the bone, and remain implanted in the body to stabilize the fracture.

In another embodiment, there is disclosed a method of reducing and stabilizing a bone fracture, the method comprising contacting a fractured bone with an implantable medical device as described herein. In particular, the method comprises attaching to a fractured bone a screwless bone plating system comprising at least one clamp comprising: a top portion; a bottom portion, and an attachment for securing the clamp to a bone without drilling a screw into the bone. In this method, the clamp has functionality in that it assists in reducing the fracture by realigning the fractured ends of the bone, and stabilizing the fracture by tightening the clamp to the bone, such that the clamp remains implanted as part of the permanent implant construct.

Aside from the subject matter discussed above, the present disclosure includes a number of other exemplary features such as those explained hereinafter. It is to be understood that both the foregoing description and the following description are exemplary only.

### BRIEF DESCRIPTION OF THE DRAWINGS

FIG. 1A is a schematic of a clamp according to an embodiment with a top screw that compresses the clamp against the bone; FIG. 1B is a similar clamp (with teeth) that compresses the clamp against the bone using a different mechanism.

FIGS. 2A, 2B and 2C are schematics of various unibody or monoblock clamps that are snapped onto bones and held in place by compression. These clamps shown channels on the top surface that allow bone plates to translate along the channel when used. FIG. 2B is shown with teeth for attaching to bone.

FIG. 3A is an additional embodiment of the clamps shown in FIGS. 2A-2C. These show dual clamps permanently connected by a bone plate. FIG. 3B shows the dual clamp placed over a single clamp that is positioned at the fracture site.

FIG. 4A is a schematic of a bone plate that can be connected to a clamp, such as the clamps shown in FIGS. 1 and 2. Unlike the embodiment shown in FIG. 3A, FIG. 4B shows the bone plate is removably connected to the clamps, for example by a screw applied through the plate and clamp.

FIG. 5 is a schematic of a bone plate that can be connected to a clamp according to an inventive embodiment.

FIG. 6A is a schematic of a bone plate attached to the top portion of a single clamp. The bone plate shows channels that allow the plate and the clamp to move relative to each other. FIG. 6B shows a plate attached to two clamps, located on opposite sides of a bone fracture.

FIGS. 7A and 7B are schematics of double clamp connection plates showing the clamps in an open position, for attachment to bones of variable thickness. In an embodiment, this double clamp can be connected to another clamp, such as shown in FIG. 1B, such that the clamp of FIGS. 7A and 7B is put on top of the single clamp of FIG. 1B to enhance screwless fixation of a bone.

FIGS. 8A to 8D are schematics of compression plates, such as those that can be used to fix ulna fractures.

FIGS. 9A to 9E are schematics of radial neck plate according to various inventive embodiments.

FIGS. 10A to 10C are schematics of distal fibular according to various inventive embodiments.

FIGS. 11A to 11C are schematics of distal fibular according to various inventive embodiments.

FIGS. 12A to 12D are schematics of fibular plate and clamps according to various inventive embodiments.

FIGS. 13A to 13C are schematics of distal clavicle according to various inventive embodiments.

FIGS. 14A to 14D are schematics of clavicle plates attached to clamps according to various inventive embodiments.

FIGS. 15A to 15E are schematics of phalange) plates according to various inventive embodiments.

FIGS. 16A to 16D are schematics of phalangel base plates according to various inventive embodiments.

FIGS. 17A to 17D are schematics of unibody or monoblock olecranon plates according to various inventive embodiments.

FIGS. 18A to 18E are schematics of olecranon plates according to various inventive embodiments.

FIGS. 19A to 19D are schematics of olecranon plates according to various inventive embodiments.

FIG. 20 is a schematic of a front view of a distal radial plate according to one inventive embodiment.

FIG. 21 is a schematic of a front view of a distal radial plate according to an inventive embodiment.

FIG. 22 is a schematic of the back view of a distal humerus plate according to one inventive embodiment.

FIG. 23 is a schematic of a distal humerus plate according to one inventive embodiment.

FIG. 24 is a schematic of a front view of a proximal humeral plate according to an inventive embodiment.

### DETAILED DESCRIPTION

As used herein, the phrase “screwless bone plating” refers to a system that attaches at least one clamp to a bone without requiring the clamp be secured to the bone by a bone screw. Screws may be used to attach plates to clamps, and plates to plates; however, screws are not required to attach the clamps to the bones. In some embodiments, once attached, the clamps and/or plates may be additionally secured to the bone with a screw, but it is not required.

The screwless bone plating system described herein allows quick fastening and unfastening of the clamp, or a clamp and attached plate, to a bone.

As used herein, the term “ratchet” refers to a round gear or linear rack with teeth, and a pivoting, spring-loaded finger that engages the teeth. When the teeth are moving in the unrestricted forward direction, for example to tighten a clamp to a bone, the finger easily slides up and over the teeth, with a spring forcing it into the depression between the teeth as it passes the tip of each tooth. When the teeth move in the opposite (backward) direction, however, the finger will catch against the edge of the first tooth it encounters, thereby locking it against the tooth and preventing any further motion in that direction. Thus, the clamp will not become lose on the bone.

As used herein, the term “snap fit” refers to the ability of a clamp to have direct clamp to bone attachment by simply snapping the clamp onto a bone. Attachment to the bone does not require screws to be screwed into a bone, or additional clamping mechanism, however, neither are precluded from a snap-fit body when additional robustness of the attachment or stability of the bone is required.

As used herein, the term “degrees of freedom” refers to the ability of the clamp and/or plate to translate and/or rotate relative to each other or relative to the bone on which they are attached. For example, when translating relative to each other, the plate can move up and down the clamp; left and right on the clamp; and forward and backward on the clamp. When rotating relative to each other, the plate and clamp can tilt forward and backward; swivel left and right; or pivot side to side. As used herein, the term “at least three degrees of freedom,” means that the clamp and/or plate can translate and/or rotate in at least three of the foregoing manners, including any version of translation or rotation described herein.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly.

There is disclosed an implant system that can be used to stabilize and realign bone fractures or osteotomies that correct bone deformities. This system is unique to other implant systems in that it not only provides stabilization of bone fractures and osteotomies, but it allows the user to freely manipulate bones, distract or compress osteotomy and fracture sites thru the implant themselves thus lending to unique functional capabilities not currently available in today's implants. What is also unique is that the stabilization of osteotomies or bone fractures, compression or distraction of bone can be accomplished without the use of bone screws.

In the disclosed inventive system, clamps of various shapes and sizes are used not only to grab and position the bone but to act as part of the definitive stabilization. Clamps can be used independently or integrated into plates in various ways to enhance functionality specific to a particular fracture, osteotomy or bone geometry. When attached to plates, clamps can be fixed or have at least three degrees of freedom. Clamps can be attached above or below a plate, or can be integrated or built in as part of the plate to maintain a low profile. When clamps are used independent of plates there are options to supplement stabilization of bone by connecting plates to the clamps, thus adding modularity to the system. Also when clamps are used independent of themselves there is also opportunity to provide further screwless supplemental stabilization of bone by designing plates to fit around clamps. As discussed in more detail below, various clamps designs allow for fixation of bones having variations in width and geometry.

As shown and described herein, the novel implantable medical device include plates having combinations of fixed or mobile clamps that can translate, swivel or rotate in various ways in relation to the plate so as to allow for bone movement thru the implants. This functionality allows the surgeons to achieve their goals of bone realignment. By incorporating functionality into the implants, surgical techniques are simplified. The system also allows for fast applications of plates to bones without the need for bone screws or wires to hold their position. It allows for quick and easy repositioning of implants. By integrating bone realignment into the implant this system becomes more surgically efficient in that simultaneous plate application is being performed as the bones are being realigned.

Clamps and plates in this invention also improve efficiency in surgical technique, by having the clamps center the plates squarely on the bone they are intended for.

Furthermore, in the disclosed implant system, interfragmentary screw fixation can be transitioned to a more stable clamp fixation. If bone screws are desired as part of the stabilization, the current invention allows for more stable configuration of screw patterns, whether screws are placed across clamps or plates. Perpendicular screw patterns can be achieved thru the clamp or plates.

The system may allow for decreased healing times and non-union rates in fractures and osteotomy sites by at least the following:

1. Preserving Endosteal Blood Supply when used in a screwless fashion;

2. Compressing osteotomies or fracture sites; and

3. Using clamps independently, which lead to shorter constructs that require less periosteal stripping, hence preserving periosteal blood supply.

In view of the foregoing, there is disclosed herein a series of orthopedic implants designed to assist an orthopedic surgeon in fracture reduction, and stabilization, which can be incorporated into the final implant construct. The orthopedic implants described herein reduce and simplify the transition from bone fracture reduction to fixation by allowing the inventive clamps to assist in centering the hardware centrally on the fractures bone(s).

There is described an implantable medical device for restoring bone alignment and stabilizing a fracture, comprising clamps and plates that are fundamentally different than existing bone plating systems. On the most basic level, the plates described herein have functionality have changed so they participate in grabbing fracture ends thru the use of clamps and allowing the surgeon to use them to realign bones.

In one aspect, the clamps described herein can be positioned based on a variety of factors, including but not limited to (1) surgical exposures, (2) areas that minimize soft tissue irritation, and (3) areas where they can sufficiently envelope the bone so as to sufficiently main compressive forces.

In certain embodiments, the clamps described herein are fixed and built into the plate to lower profile. In other embodiment, the clamps are attached through a fastener, such as a posts. This embodiment allows the resulting structure to have at least three degrees of freedom, and thus allow for increased fracture maneuverability to improve ease of fracture reduction. As evident, in the implantable medical device described herein, plates and clamps could both be modified to allow for clamp movement in the system.

The ability for the clamps to move is beneficial as it allows the clamps to be used as stand-alone devices to reduce and stabilize fractures. For example, they can take the place of interfragmentary screw fixation. In one embodiment, they can be designed to have windows for viewing fracture healing on X-ray. Supplemental plates can be built around clamps to increase fracture stability when they were used as the primary fixation device.

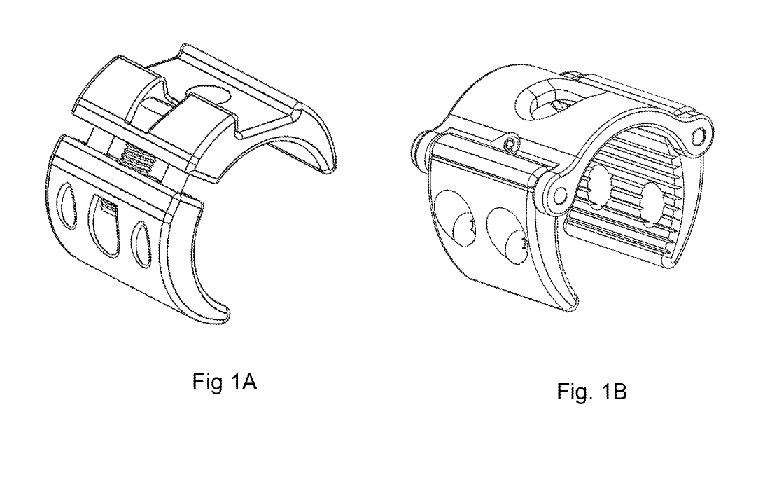

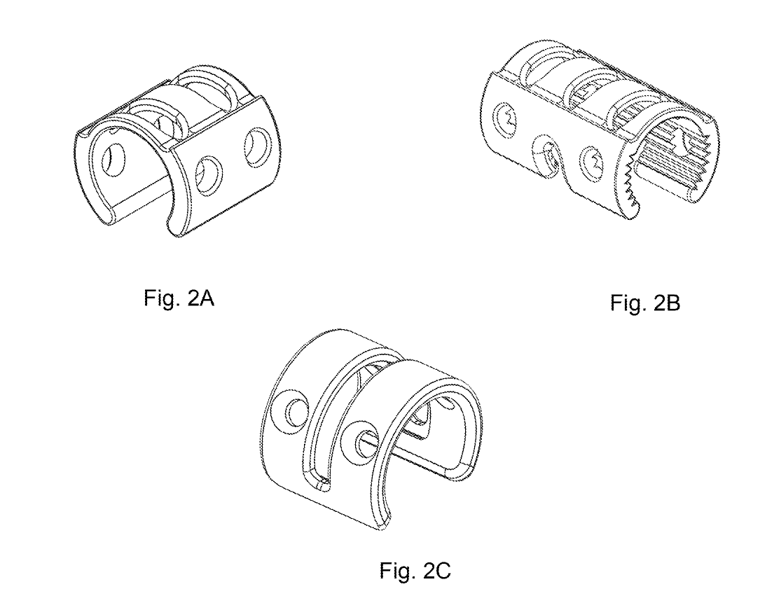

There is disclosed an implantable medical device for restoring bone alignment and stabilizing a fracture, comprising a screwless bone plating system for contacting at least a portion of a bone. In one embodiment, the screwless bone plating system comprises at least one clamp comprising: a top portion; a bottom portion, and an attachment for securing the clamp to a bone without drilling a screw into the bone. FIGS. 1 and 2 show The Inventor has discovered that the clamps disclosed herein are capable of realigning a fracture when applied to the bone, and remains implanted in the body to stabilize the fracture.

The described attachment may comprise one or more adjustable clamping members that engage the bone and secure the clamp to the bone with at least one fastener. For example, the fastener may comprise at least one set screw, pin, peg, ratchet, or combinations thereof, which fasteners may be configured to maintain, adjust, or both maintain and adjust, the pressure that the one or more adjustable clamping members apply to the bone. For example, FIG. 1 shows a clamp according to an embodiment with a top screw that compresses the clamp against the bone FIG. 1B is a similar clamp, with the additional component of grooves or teeth on the clamping surface, which help the clamp grip the bone and surrounding soft tissue.

The bottom portion of the clamp which attaches to the bone may comprise a singular piece (mono-block), such as metal-based, a snap-fit body that snaps onto a bone. In one embodiment, the snap-fit body comprises a metal or alloy that is in shape of the bone to be contacted that fits over a bone, and remains fixedly attached to the bone by compressive forces. FIGS. 2A to 2C show various clamps made from a singular piece of metal, and that can be snapped onto bones and held in place by compression. These clamps shown channels on the top surface that allow bone plates to translate along the channel when used. FIG. 2B is shown with teeth for attaching to bone and/or soft tissue.

In one embodiment, the described clamp comprises a top portion having at least one hole or channel extending there-through, the hole or channel being adapted to receive a screw for attaching a bone plate to the clamp; and a bottom portion comprising a metal or alloy containing body that snaps onto a bone, or that remains fixedly attached to a bone by compressive forces.

One aspect of the disclosure provides for at least one plate that is attached to at least one hole or channel in a fixed and unmovable position. Alternatively, the disclosed plate may be attached to the channel, which allows the plate to rotate with up to three degrees of freedom, such as moving up and down, side to side, or swivel, relative to the bone and or clamp, prior to the plate be fixedly secured to the clamp with one or more screws.

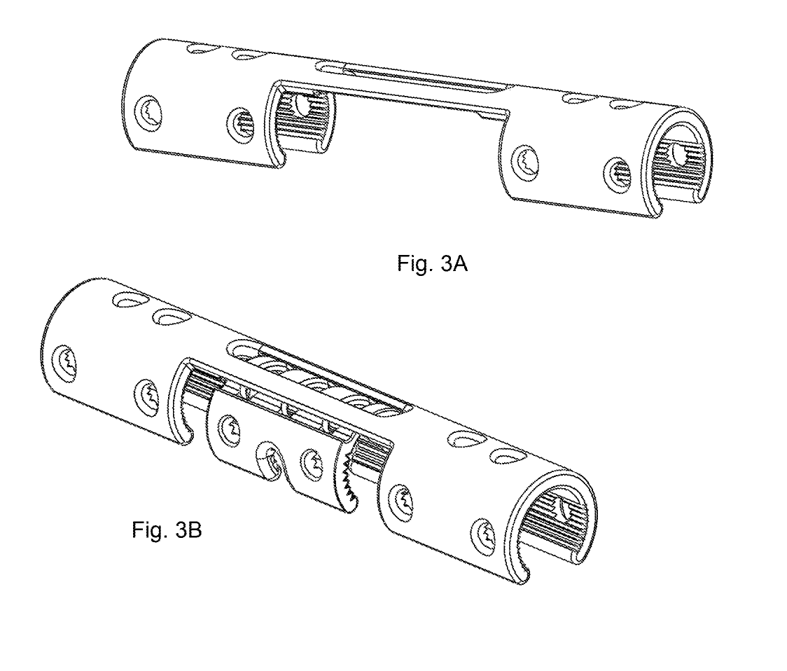

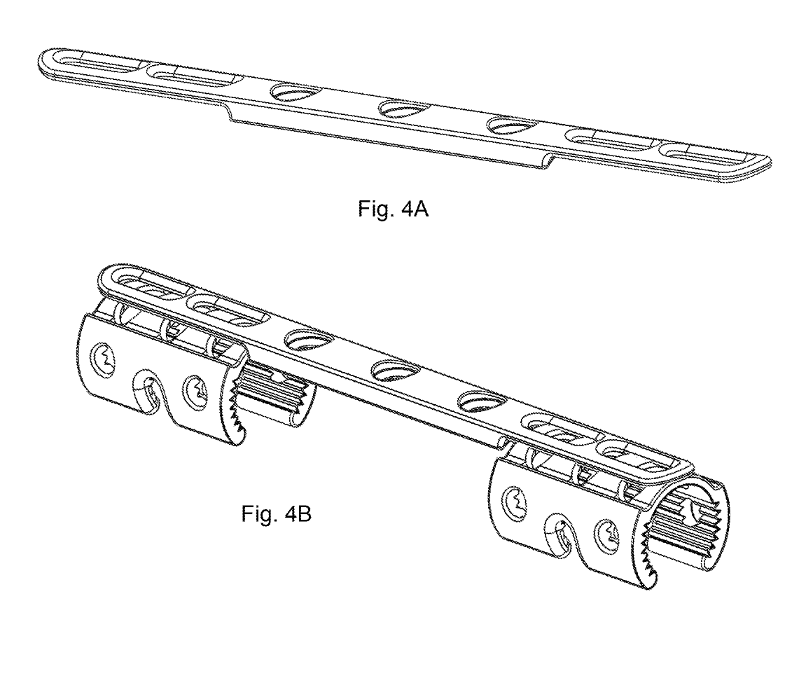

An embodiment has been described in which only one singular piece (mono-block) is attached directly over the fracture. There are additional embodiments in which at least two clamps are attached on opposite sides of a bone fracture, with at least one bone plate spanning the bone fracture and attached to the two clamps. For example, FIGS. 3A and 3B, show these embodiments when the plate is attached in a fixed or permanent position. FIG. 4B shows a different embodiment when the plate (FIG. 4A) is removably attached to the plate.

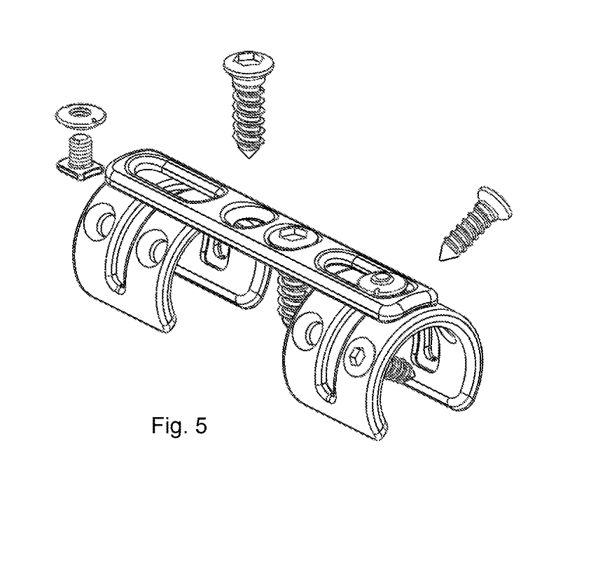

When the implantable medical device described herein comprises a plate attached to a clamp it is typically attached to the top portion of the clamp at a hole or channel extending there-through, and being adapted to receive a screw or connection post to attach the clamp to the bone, to a plate, to another clamp, or any combination thereof. See for example, FIG. 5, showing how the bone plate can be attached to two clamps. Alternatively, (and not shown in FIG. 5), the bone plate may be positioned against the bone and underneath the clamp, such that the clamp secures the bone plate to the bone when tightened to the bone.

In one aspect of the invention, the clamp comprises multiple parts, such as two parts, which can be placed on opposite sides of a bone, particularly at a fracture sight. Then the two opposing sides are connected to each other, such as by a tightening a screw. As the separate sides are brought into contact, compression forces occur on the bone, thereby stabilizing the fracture site. The fracture site is both reduced and stabilized with a clamp without any screw touching the bone. This also allows the clamp to be translated up and down the bone prior to final tightening of the clamp.

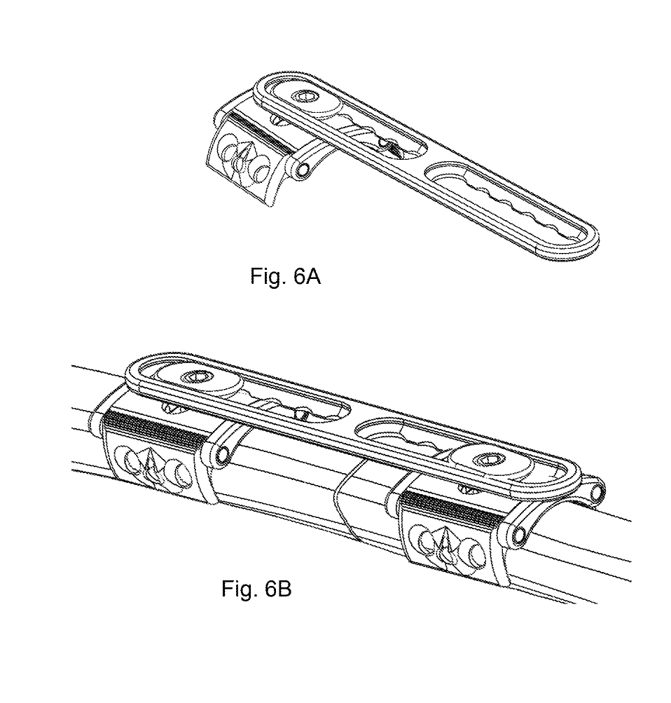

In some aspects, the plate is attached to the clamp in a fixed and unmovable position. In other aspects, the plate is attached to a channel in the clamp, which allows the plate to translate up and down the channel prior to the plate being fixedly secured to the clamp with one or more screws. FIGS. 6A and 6B show a plate attached to a singular clamp via a channel in the plate (FIG. 6A), and to dual clamps via a channels in the plates (FIG. 6B).

There are many variations of the plates and clamps, which lead to significant maneuverability and freedom. For example, the plate may have attached thereto two clamps, both being fixed, or one being fixed and the other rotating with at least three degrees of freedom, or both rotating.

As shown, the disclosed devices have versatility and scalability to allow the surgeon to daisy-chain multiple plates when necessary to set long or multiple fractures. Thus, in one embodiment there is disclosed a modular fracture fixation system comprising two or more implantable medical devices as described herein, wherein the two or more implantable medical devices comprise multiple clamps that can be positioned along the length of a bone, and proximal to a bone fracture. This modular fracture fixation system may further comprise at least one bone plate attached to one or more of the multiple clamps.

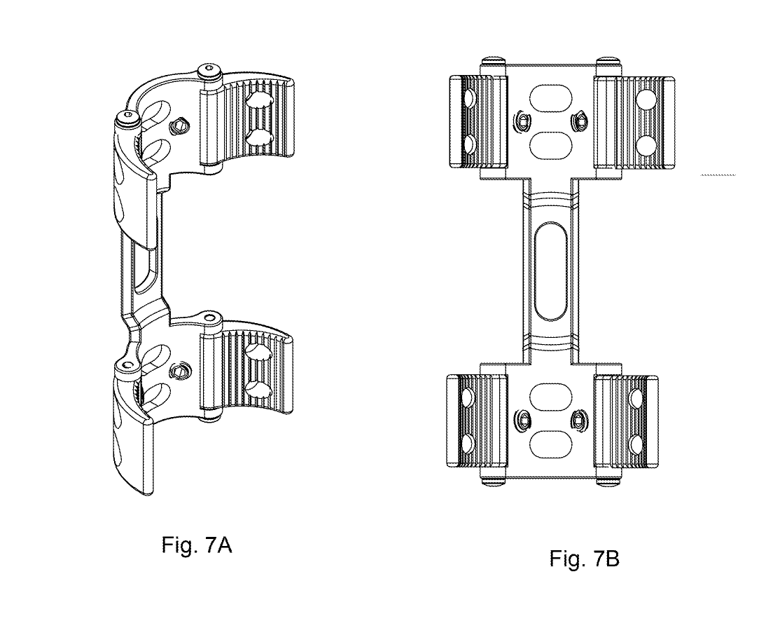

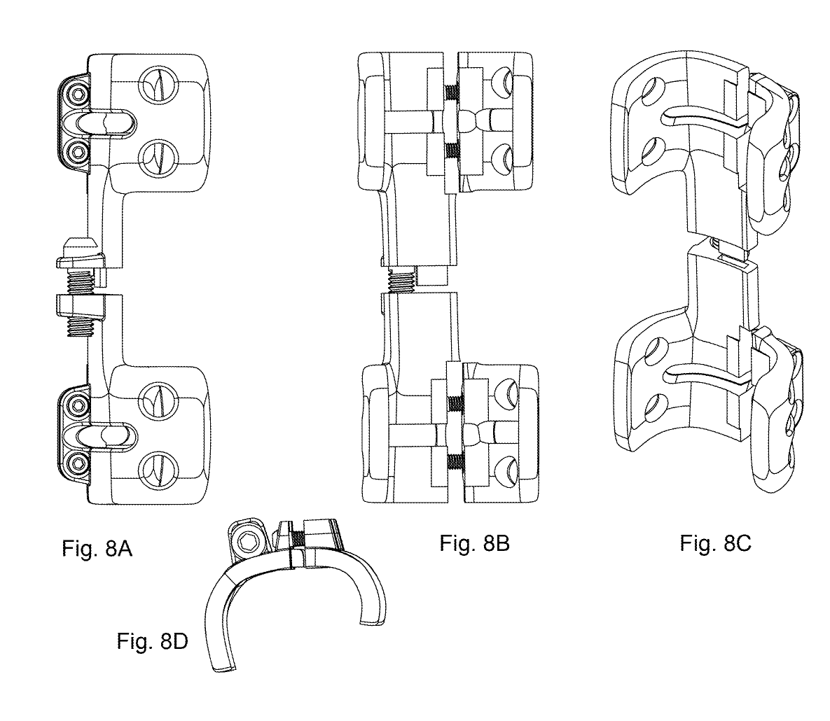

In another embodiment, the implantable medical device described herein comprising a screwless bone plating system for contacting at least a portion of a bone. The screwless bone plating system comprises at two clamps for attaching to a bone, the clamps being located on opposite sides of a bone fracture and each comprising: a top portion comprising at least one hole or channel extending there-through, the hole or channel being adapted to receive a screw or connection post for attaching a bone plate to the clamp; and a bottom portion comprising an attachment for securing the clamp to a bone without drilling a screw into the bone. In this embodiment, the at least one bone plate is attached to the two clamps and span the bone fracture. Like the previous embodiment, the two clamps are capable of realigning the fracture when applied to the bone, and remain implanted in the body to stabilize the fracture. FIGS. 7A and 7B show the underside of dual clamps that are connected with a plate. Similar compressions plates are also shown in FIGS. 8A-8D, with 8D being a side view of the device showing the relatively low profile characteristics of the device. These types of implantable devices could be used to reduce and stabilize an ulna fracture, or also to compress an osteotomy site.

In one embodiment of this implantable medical device, the bone plate is permanently attached to the clamps in a fixed and unmovable position. In another embodiment, the clamps are movably attached to the plate, such as being able to rotate 360 degrees. In another embodiment, the top portion of the plate may comprise a channel, and a screw that connects the channel in the plate to the channel in the clamps such that the plate can translate and rotate with up to three degrees of freedom prior to the plate being fixedly secured to the clamps with one or more screws.

As described for the single clamp and single plate, an implantable medical device comprising a screwless system that has multiple plates and clamps, may also have channels in the plates and clamps, which allow the plates and the clamps to translate to up and down the channels in the clamp and the plate prior to the plate being fixedly secured to the each other with one or more screws. This provides a lot of flexibility in positioning the plate over a fracture, which is currently not available in existing bone plating systems. In some embodiment, plates have the ability to maneuver fracture ends in multiple planes.

In one embodiment of this implantable medical device, the disclosed clamps could be designed for small bones to keep them low profile. This system can be used as a screwless system preserving endosteal blood supply, or when screws are placed the configurations that can be achieved are more biomechanically stable.

When bone plates are used in conjunction with the described clamps, they are generally attached to the top portion of the clamp. However, it is possible that at least one bone plate positioned against the bone, e.g., underneath the clamp. Then at least one clamp can be applied over the plate, which will secure the bone plate to the bone.

In another embodiment, the proximal portion of the bottom surface of the bone plate curves upwardly to form a concave bottom surface configuration at the proximal portion of the plate segment when viewed from the proximal end, such that the concave bottom surface configuration is adapted to fit a curved contour of a bone.

In another embodiment, there is disclosed a method of reducing and stabilizing a bone fracture, the method comprising contacting a fractured bone with an implantable medical device as described herein. In particular, the method comprises attaching to a fractured bone a screwless bone plating system comprising at least one clamp comprising: a top portion; a bottom portion, and an attachment for securing the clamp to a bone without drilling a screw into the bone. In this method, the clamp has functionality in that it assists in reducing the fracture by realigning the fractured ends of the bone, translating at least one clamp to the fracture site to provide stabilization, and stabilizing the fracture by tightening the clamp to the bone when at the fracture site, such that the at least one clamp translated to the fracture site remains implanted as part of the permanent implant construct.

In one embodiment of the method described herein, the clamp is attached to the fractured bone with one or more adjustable clamping members that are locked into place by at least one fastener selected from a set screw, pin, peg, ratchet, or combinations thereof. These fasteners are configured to maintain, adjust, or both maintain and adjust, the pressure that the adjustable clamping members apply to the bone.

In one embodiment, the clamp is attached to the fractured bone by snapping the bottom portion of the clamp to a fractured bone, wherein the clamp remains fixedly attached to the bone by compressive forces. As previously described, clamps may be used as a stand-alone device to reduce and stabilize a fracture, or may be used in conjunction with one or more bone plates. When used with bone plates, the plates may be positioned directly on the bones, and held in place by the clamp(s), or the bone plates may be attached to the top portion of the clamp. In another embodiment, bone plates may be positioned both below the clamp (in direct contact with the bone) and attached to the top portion of the clamp.

As mentioned, by virtue of channels located in the clamp, in the plate, or both in the clamp and the plate, the surgeon has the ability and flexibility to quickly translate the clamps and the plates to an optimum position for the most effective fracture stabilization prior to the plate be fixedly secured to the clamp.

One versatile aspect of the disclosed method is in the ability to attach multiple clamps along a bone, such as if to fix multiple fractures. The clamps themselves may be used to reduce the fracture by realigning the fractured ends of the bone, translating at least one clamp to the fracture site to assist in bone stabilization, and stabilizing the fracture by being tightened to the bone at the level of the fracture site. Alternatively, at least one bone plate may be attached to the clamps such that the bone plate spans the bone fracture to enhance stabilization of the fracture.

There is also disclosed a method for reducing multiple comminuted fractures, such as three and four part, by positioning clamps to incrementally restore fractures, such as on a proximal humerus fracture. When fragments have soft tissue, the clamp attachments may have clamp arms with teeth designed to also grab on to soft tissue (proximal humerus).

As shown, the inventive implantable medical devices and methods of using are designed to eliminate time spent making sure that plates are centrally located on bones. In one embodiment, the disclosed plates allow for clamp and perpendicular locking screw fixation of the plate. This increases the biomechanical stability of the construct and diminishes the amount of screws needed to secure fixation, as well as allows for shorter plates to be applied. This feature allows for reduced incision, exposures, soft tissue trauma, decreased operating room and Fluoroscopic times.

The disclosed plates allow for easier adjustments of plates both proximally and distally on the shaft of the bone, eliminating the need for screw removal or loosening of clamps to adjust plates. The clamps will continue to maintain fracture reduction and allow the plate to remain centrally located during translation of the plate on the bone. This feature is not only helpful in centering the plate in mid-shaft fractures, but is also a major advantage in placement of hardware at the terminal ends of a bone or periarticular plating.

The clamps integrally attached to the disclosed plates are used in the fracture reduction process and become a permanent part of the implant. This feature also decreases operating room and fluoroscopic times by facilitating the transition between fracture reduction and application of hardware. By using the disclosed plates, fracture reductions no longer have to be lost when clamps are repositioned to allow for placement of hardware.

In addition, traditional fixation techniques such as inter-fragmentary screw compression can still be performed with the disclosed devices and system.

In one embodiment, the system incorporates low contact plating designs. In addition, the friction fit design at the screw plate interface speeds up the process of locking screws. In another embodiment, certain fractures maybe amenable to clamp fixation alone.

The disclosed bone plating system allows a surgeon to adjust plates proximally or distally on the shaft of a bone after fracture reduction. By using the disclosed bone plating system, application is also simplified in that there is no need for screws, or wires, such as Kwires to be removed or surgical instrument-placed clamps adjusted to move the implant. Shorter implants should translate into smaller incisions and exposure and subsequently less soft tissue trauma. Ultimately, these implants are designed with the orthopedic surgeon in mind, to reduce operating room and fluoroscopic times, limit incisions and take some of the unplanned maneuvering out of fracture fixation.

While the implantable medical devices disclosed herein are meant to limit the length of plate needed, the system is modular and in certain circumstances, allow for the plates to span the entire length of a long bone. In certain aspects, the described clamps may act as a substitute for inter fragmentary screw compression if applied directly at the fracture site.

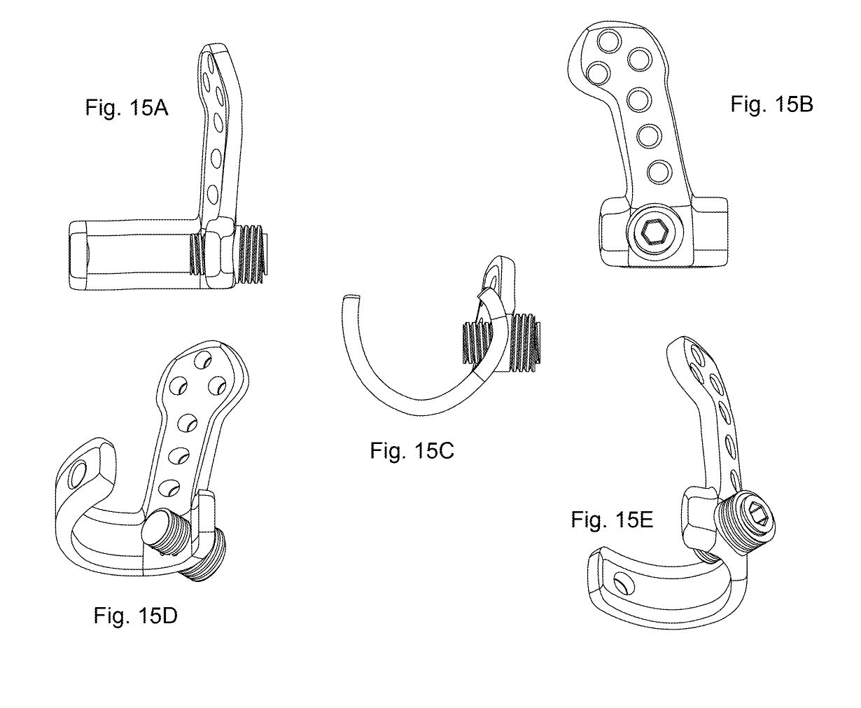

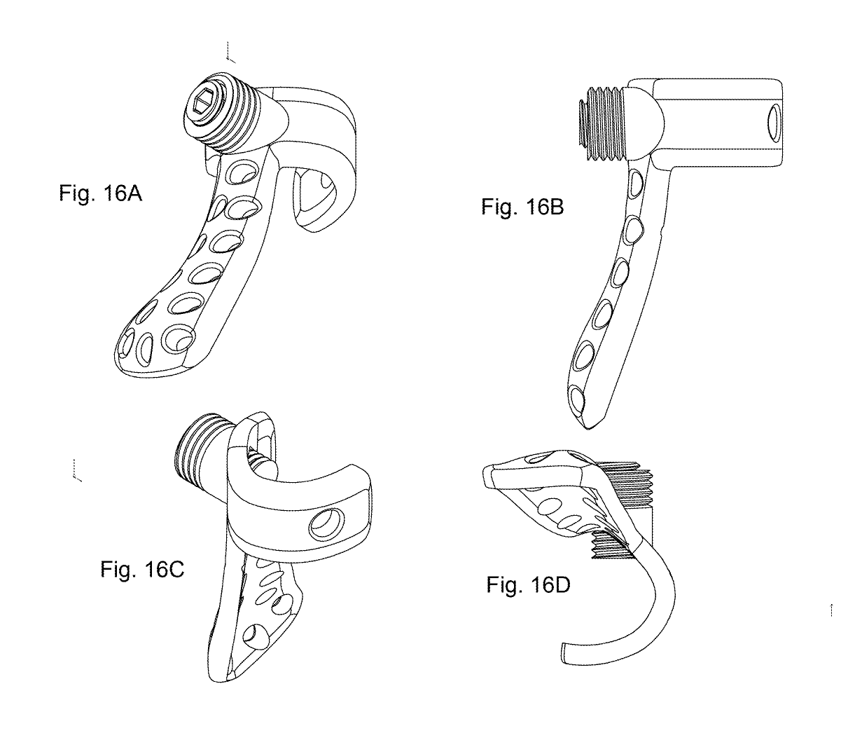

In various embodiments, the versatility of the clamps and plates described herein can be made into various designs, including but not limited to fixing smaller hand fractures. Non-limiting examples of the inventive clamps and plates that can be used in these applications are shown in FIGS. 15 and 16. In addition, the versatility of the clamps and plates described herein can be used to fix long bone type fractures, such as common bones in the arm, including the humerus and the forearm bones, i.e., the radius and ulna radius. It is appreciated that the various designs are fracture specific.

In one embodiment, there is disclosed a fixed plate, referred to as a double fixed clamp plate. In this embodiment, the clamps are not allowed to swivel in relation to the rate in this design. Fractures are exposed and manually reduced using standard techniques. The plate and fixed double plate constructing centered over the fracture, with one clamp positioned above and one clamp position below the fracture site. In this embodiment, the plate is snapped into place and the clamp inserter handles are hand tightened subsequently tensioning clamps. The clamps can have locking screws applied across the bone and clamp allowing for maintenance of clamp tension and further securing of implant.

Dynamic compression of the fracture site can be done through screw holes in the rate if required. Further addition of friction screws/locking screws and cortical screws continue to stabilize the fracture and implant. This plate does not preclude inter-fragmentary screw compressional the start of the procedure if desired.

In another embodiment, there is disclosed a swivel plate, referred to as a double swivel clamp plate, in which the clamps are allowed to swivel in relation to the plate. This added extra moment of freedom allows the plate to be applied across the unreduced fracture sites, the fracture can then be reduced as the clamps and the attached clamp inserters swivel the fracture into a reduced position. Once the fracture is reduced the clamps are locked and not allowed to swivel, the locking screws are applied across the clamps and plate allowing definitive fracture and implant stabilization. Another option for this plate would be to not only have the clamps swivel but to translate proximally or distally along the plate. Adding another degree of freedom may further aid in fracture reduction. Strategic placement of clamps proximally or distally could further increase fracture stability.

This is the most versatile of the constructs and its modular nature can allow for an entire long bone to be spanned. In this construct, the clamps are applied through in inserters above and below a fracture site. The fracture is subsequently reduced and a plate that spans both clamps and fracture site is snapped into the clamps, allowing for maintenance of reduction. Since there are no restrictions on the clamps during fracture reduction, this give the most absolute momenta of freedom during fracture reduction, however special attention must be paid to align the clamps to allow for the connecting plate to be snapped into place. Each clamp can accommodate two plates being snapped into place. The combination of multiple clamps and different length plates being used adds to the versatility of this system.

As one skilled in the art would appreciate, the bone plate described herein may be left in place permanently or removed after the fractured bone has healed. Bone plates as described herein are configured to stabilize at least one bone by being attached thereto, typically on the exterior of the bone. Generally, the bone plates should be stiffer and stronger than the section of bones on which it is attached. Non-limiting examples of suitable materials may be biocompatible materials, such as titanium or titanium alloys (specifically alloys containing aluminum and vanadium), cobalt chromium, stainless steel, plastic, and ceramic. Bioabsorbable materials (such as polygalactic acid (PGA), polylactic acid (PLA), copolymers thereof, etc.), may also be used.

The disclosed embodiment may provide an implantable medical device and improved bone plating system, as well as methods of using the same. These novel devices and methods of use are further described with respect to the following specific bone fractures, and methods of reducing such fractures, for illustrative purposes with particular reference to the Figures, when appropriate.

In one embodiment, two clamps can be used to reduce, then internally fix fractures. For example, FIG. 1A shows a clamp with a screw and two pegs; however, the flexibility of the disclosed clamps allow various configurations, for example, two screws can be used, or a screw and a peg. FIGS. 2A and 2B show alternatives to the clamps in FIG. 1 in that no mechanical aspects of the clamps exists. Rather, there is a singular block that snap onto the bone and remain in place through compression forces. As shown, with either embodiment, the clamps may be textured with teeth to grip bone and/or soft tissue surrounding the bone.

As shown in FIG. 1, the universal clamp can be applied to fractured shafts of bones, and can accommodate various bone widths. This embodiment shows a clamp that can be locked in two locations. For example, the set screw is loosened and extends to wedge up against one of the wings of the clamp. Also, this clamp can be locked from the side.

As described, plates can be built around these clamps. As shown in FIG. 1C, a fixed double clamp plate that can go on top of this clamp, and screws can be applied in a perpendicular fashion in the clamp. This shows how clamps can take the place of interfragmentary screw fixation. This Figure further illustrates a slot in the clamp align with slots in the plate to connect the two and screws can be applied thru both implants in this design.

Ulna

The following description is related to various aspects of ulna fixation using the various implantable medical device embodiments described herein, and shown in FIG. 8.

As shown in FIG. 8, one aspect of the disclosure is directed to an Ulnar Compression Plate. This plate illustrates how compression of a fracture or osteotomy site can occur thru a plate, all done screwlessly. Two fixed clamps are attached thru a plate, which is broken up into two halves, and connected thru a rail or rectangular slot and a screw. The screw that attaches both plates is 3.5 mm and will cause compression of bone when tightened. During surgery, two clamps can be applied one on each side of the osteotomy or fracture site. Screws can be perpendicular to one another and are optional in clamps.

Radius

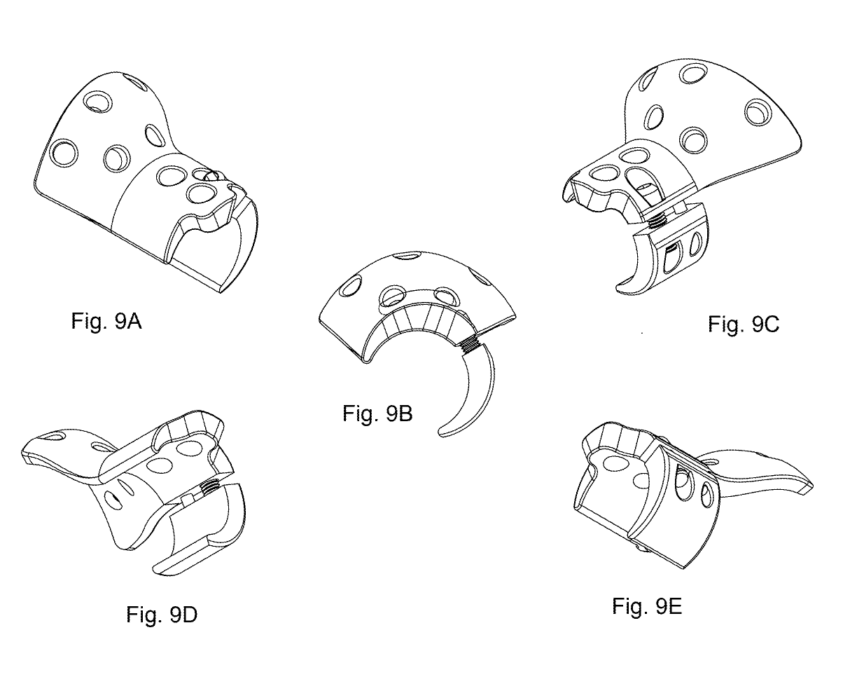

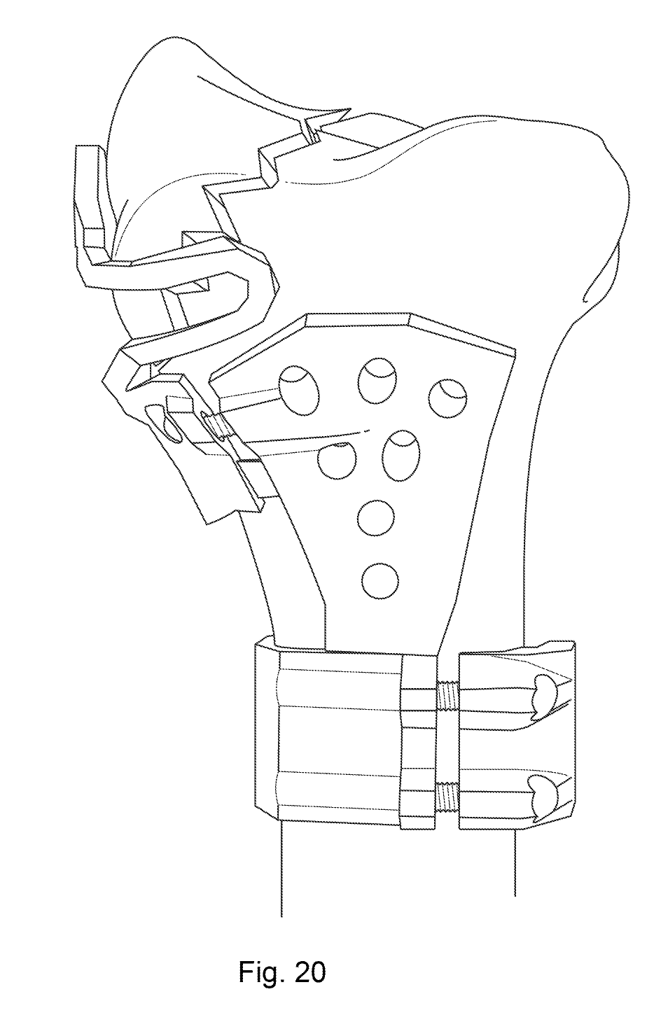

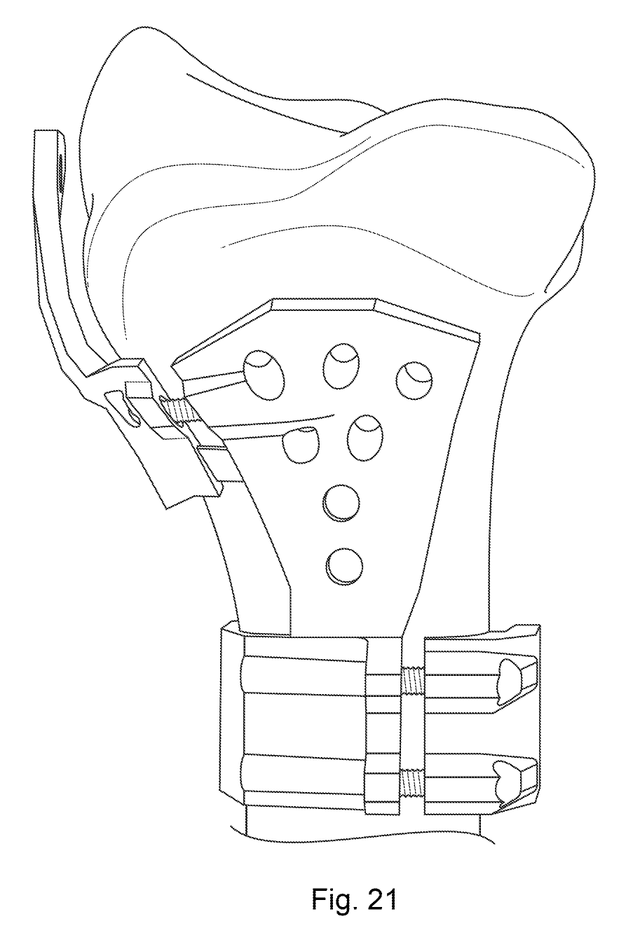

The following description is related to various aspects of radial fixation using the various implantable medical device embodiments described herein, and shown in FIGS. 9, 20, and 21.

There is also disclosed implantable medical devices that can be used to fix fractures in the radial head and neck of bones. For example, FIG. 9 shows how such a plate is specifically contoured to fit the geometry of the area, and the clamp and screw are optimally positioned given soft tissue exposure. In one embodiment, the clamp comprises at least one peg to add to further clamp stability and to keep the clamp halves from rotating in relation to one another. The proximal portion of this plate typically encompasses 100 degrees around the radial head and is applied a “safe zone” so as not to encroach on the proximal radial and ulnar joint.

The method of fixing fractures in the radial head and neck of bones may comprise reducing and restoring the radial head articular surface, with or without the use of interfragmentary screw fixation. The plate may then be clamped on the shaft of the radius and the plate position, such as height, can be easily adjusted. This clamp may be tightened by screwless distal fixation. Variable Locking Screws can be applied 3.5 mm into distal shaft, if desired, and 2.0 mm screws can be applied into radial head.

One embodiment is directed to a volar plate [e.g., the undersurface or palm side of the wrist] that provides screwless fixation of the radial plates, such as distal radial plates, which can be a low profile plate that is contoured to the distal radius. In this embodiment, the proximal clamp is attached the plate to the shaft of the radius, makes the plate become centered on the radius. The clamp is integrated into the plate to keep it low profile and may have one or more screws for tightening and fixation. The clamp further comprises at least one wing that allows for perpendicular screw fixation proximally. The plate distally stops at the epiphyseal scar to prevent hardware irritation. The distal clamp provides multiple functional benefits, including allowing for reduction of articular gapping of the surface. It also reduces radial translation of the fracture site. Screw hole in clamp allows for perpendicular screw fixation and further compression of articular surface. In one embodiment, the clamp comprises a rectangular peg for clamp support and to keep it from rotating and a 2.0 mm compression screw. The proximal clamp used two 3.5 mm screws. In addition, 2.0 mm screws distally and 3.5 mm screws proximally can be used across the plate to stabilize the fracture if desired. Design of this plate allows for rapid application and self-centering to bone without use of wires, screws, whirlybirds, while allowing for easy adjustment of the plate height/position.

The method of fixing fractures using the volar approach to distal radius comprises reducing the fracture either manually or thru plate application. A proximal clamp and plate can then applied to radial shaft, with plate height being adjusted. Distal Clamp can be tightened to reduce articular surface. Screws can then be applied, and can be variable, locking, non-locking if desired. If distal screw fixation is preferred distal clamp can be removed.

Fibula

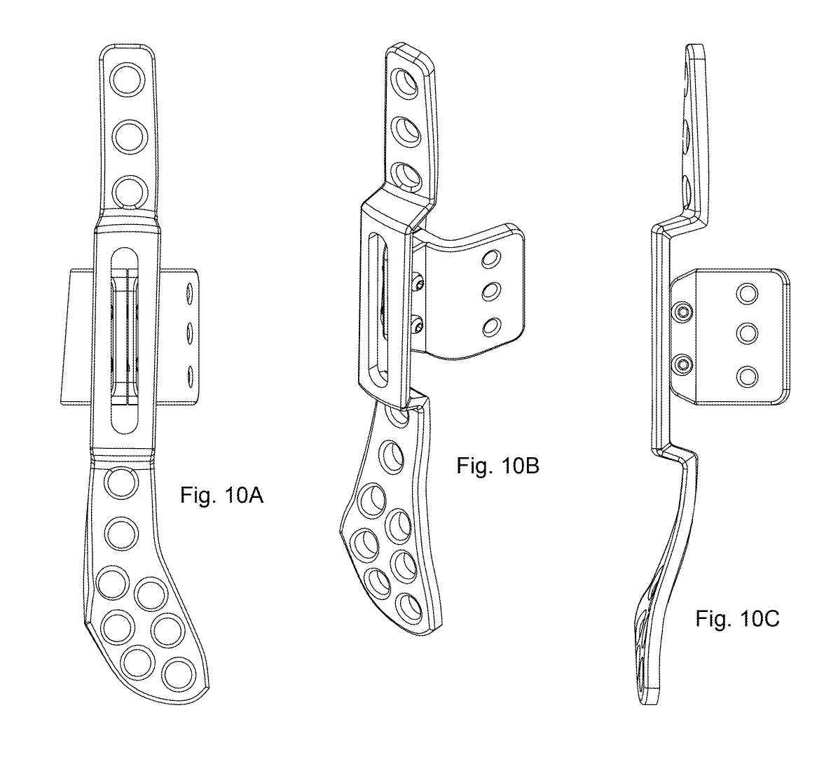

The following description is related to various aspects of fibula fixation using the various implantable medical device embodiments described herein, and shown in FIGS. 10 to 12.

With reference to FIG. 10, there is described a fibula plate with a channel that allows the plate to be translated relative to the underlying clamp. This allows for optimum placement of the plate relative to the fracture site.

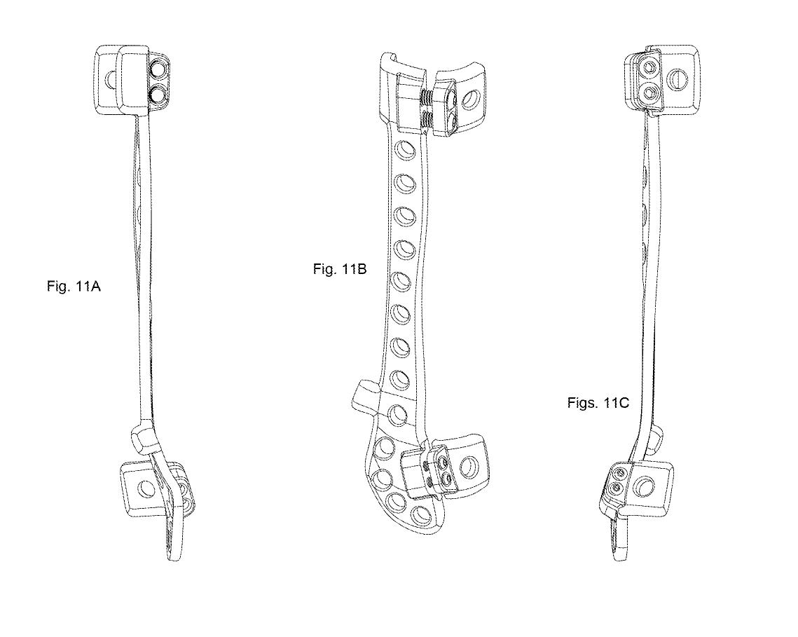

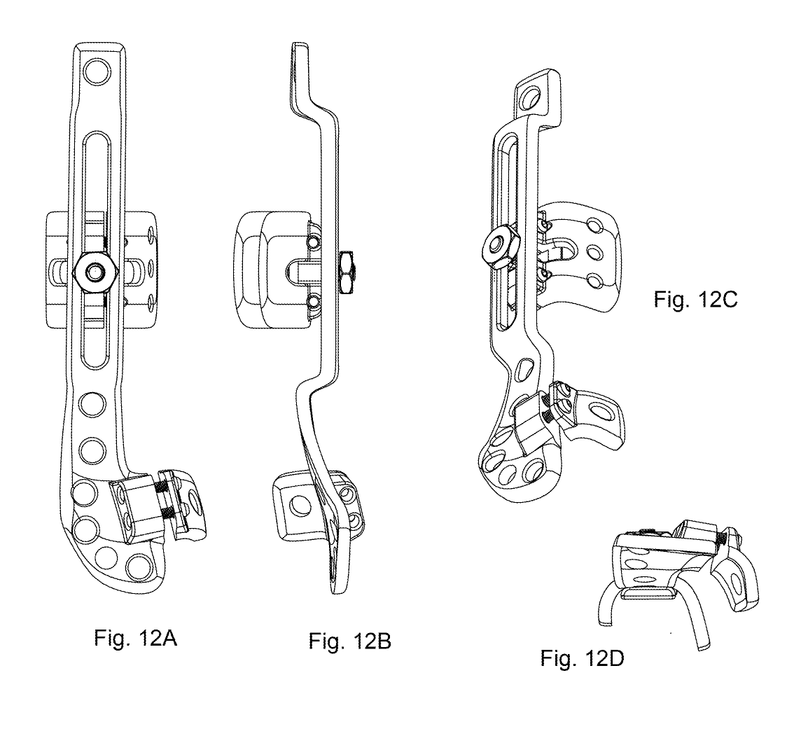

In another embodiment, there is described a fibula plate having two fixed clamps built therein to make it low profile. See FIG. 11. Alternatively, the plate may be attached to a universal clamp, such as one shown in FIGS. 1 and 2, to allow the plate to move with three degrees of freedom. See for example, FIG. 12. This particular plate has a slot to allow the clamp to move proximally or distally. The clamp can rotate and swivel around it's attachment post. Clamp position can be locked in relation to bone by using the two screws to tighten the clamp down. The clamp can be locked in relation to the plate by tightening the nut on the top of the clamp.

In one aspect, the method of using this system may comprise reducing the fracture with an interfragmentary screw. A plate can then be applied for screwed or screwless buttress support. Another approach would be to grab the distal fragment with the plate thru the distal clamp. Once the fracture is reduced, a proximal clamp could be tightened, followed by application of a distal clamp. In another embodiment, a plate is applied after the fibular clamp has stabilized a fracture. This embodiment allows for additional screwless fixation, such as by using one or more clamps. Biomechanically more stable screw configurations can be achieved (perpendicular to one another)

Another method of applying this clamp may comprise applying a first clamp to the distal fibula, which is held tensioned by surgical instrument. Next, a proximal clamp is applied proximally to shaft of fibula and held tensioned around bone by surgical instrument. Fracture reduction will then occur by manipulating the applied clamps. Once fracture reduction has occurred the proximal clamp can be translated to fracture site and further interfragmentary screw fixation could be performed if desired. Clamps can subsequently be tightened via screws with the proximal clamp being fixed to plate by a tightening nut.

In one embodiment, there is disclosed a fibular clamp which is designed to contour around the distal fibula/lateral malleolus region. Because it is contoured, the size, shape and orientation (e.g., right and left implants), are specific to the fracture it is fixing. The clamp allows for interfragmentary screw fixation and has three screw holes to allow for three 2.5 mm screws to be placed in an anterior to posterior direction.

In this embodiment, the fibular clamp can be used as a tool for reduction of a fracture by initially being applied to one of the fractured ends of a bone. The clamp can then be used to manipulate the fracture into reduction and when reduction is achieved the clamp can be translated to the fracture site and the clamp tensioned thru at least one screw. Initial clamp tensioning and repositioning can be done through a surgical instrumentation. In addition, clamp tightening can occur via an anterior to posterior direction.

Clavicle

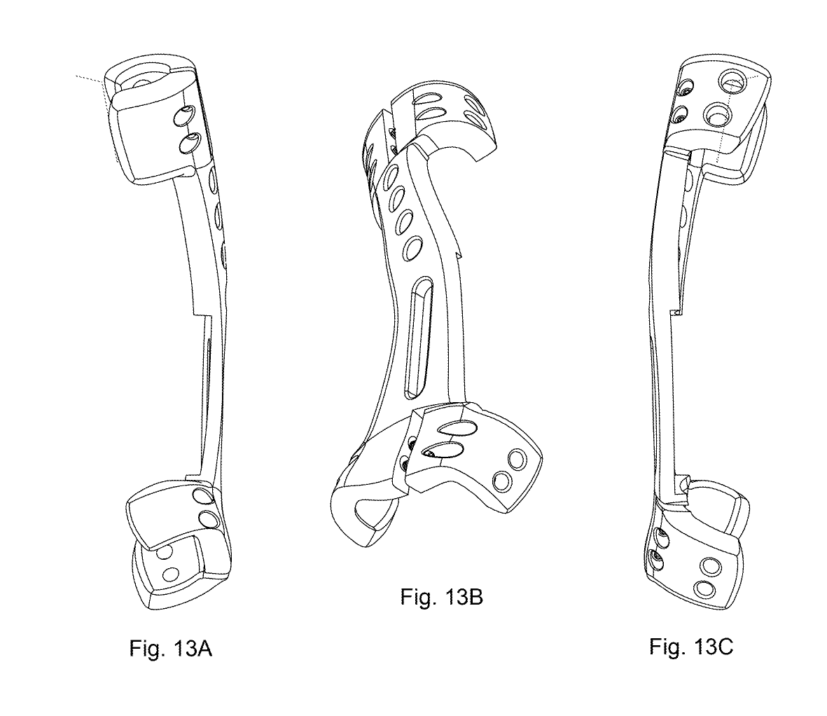

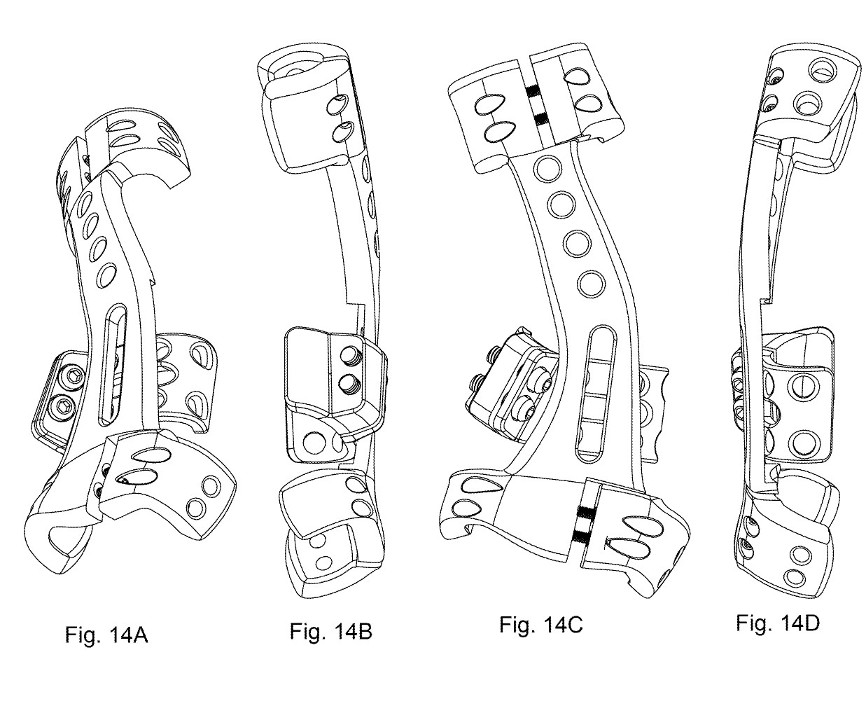

The following description is related to various aspects of clavicle fixation using the various implantable medical device embodiments described herein, as shown in FIGS. 13 and 14.

In one embodiment, there is discloses a clavicle clamp that can be used to hold one end of a fracture site so as to participate in fracture reduction. Once the fracture is reduced, the clamps described herein may be applied to fracture site to provide screwless fixation. The described clamp and surgical instrument are designed to allow for rapid application of plate to bone without the use of screws, Kwires and subsequently can be easily repositioned. However, once attached, the clamp and plate structure can be additionally secured with one or more screws, if necessary.

FIGS. 13 and 14 show distal clavicle clamps, clavicle plates, and clavicle plates with attached clamps, respectively. The Figures of the distal clavicle clamps show that the screw hole geometry allows one to place screws on both sides of a fracture in two different planes. For example, if one uses the Anterior to Posterior trajectory screw holes then one can continue to avoid neuro vascular structures. Superior to inferior (top to bottom) can be applied giving additional fixation, allowing for two screws to be applied to each side of a fracture site in a perpendicular plane to one another. The screws used to tighten the clamp can be applied posteriorly so as to keep the clamp low profile and better accommodate supplemental plate fixation. In one embodiment, there is a fracture viewing window in the clamp.

Supplemental plates can be attached both with and without clamps, and can be applied above a clamp or can be used independently. These supplemental plates can be used to fix any fracture described herein and are not limited to clavicle fixation.

The disclosed plates can be contoured for the distal clavicle and can be locked, unlocked and variable locked screws. In one embodiment, the clamps on the plate may have 2.0 mm screws and again the hole closest the screw head are typically non-threaded to allow rapid positioning on bone, initial clamp tension and repositioning is always done thru surgical instrumentation. There is a slot in the plates to allow a screw to be placed from superior to inferior and thru the clamp. The holes at the end of the clavicle are angled away from the acromioclavicular joint to mitigate against screw penetration of the joint.

In one embodiment, the method for attaching the clavicle plate is based on applying the plate clamp construct simultaneously. For example, the clamps shown in FIGS. 6A and 6B can be used to fix a fracture in a clavicle plate if the plate contour is changed to match the clavicle shape, such that one clamp can be attached on each side of the fracture to reduce the fracture. A clamp could then be translated to the fracture site if desired. The clamps would be locked in relation to the bone and in relation to the plate thru the one screw on top of the plate.

Phalanges

The following description is related to various aspects of phalangeal fixation using the various implantable medical device embodiments described herein, and as shown in FIGS. 15 and 16.

In one embodiment, there is disclosed implantable medical devices that can be used to fix fractures in phalangeal bones. For example, clamps and plate are specifically contoured to be applied to the phalanx, which may be used to fix fractures at the base of the phalanx. In this embodiment, a phalangeal clamp that allows for rapid application to the phalangeal shaft and easy repositioning. For example, the clamp may have a threaded portion to allow for a surgical instrument to be attached so as to hold and position the implant.

As shown in FIGS. 15 and 16, the phalangeal clamp may be designed to be low profile, which can be used to manipulate fractures into reduction. Once the fracture is reduced, the clamp can be translated to the fracture site to act as definitive screwless fixation. There is a screw hole in the clamp so as to allow interfragmentary supplemental screw fixation if desired. The clamp can be held into position by turning a set screw that wedges against the bone. The outer surface is typically a smooth polished surface to allow for tendons to glide against.

In one embodiment, it is possible to apply two clamps to each fractured bone end so as to use for fracture manipulation, with one clamp being able to be removed if need be. These clamps can be used on a variety of bones, such as toe phalanges, metacarpals and metatarsals.

In one embodiment, there is disclosed an implantable medical device to repair distal phalangeal neck fractures. The clamp would provide proximal screwless phalangeal fixation, easy application to bone and easy repositioning.

There is also disclosed a phalangeal mid-shaft plate that allows for fixation of mid-shaft phalangeal fractures. In one embodiment, there is disclosed one clamp in the middle of the plate that can allow for all of the features described above.

Olecranon

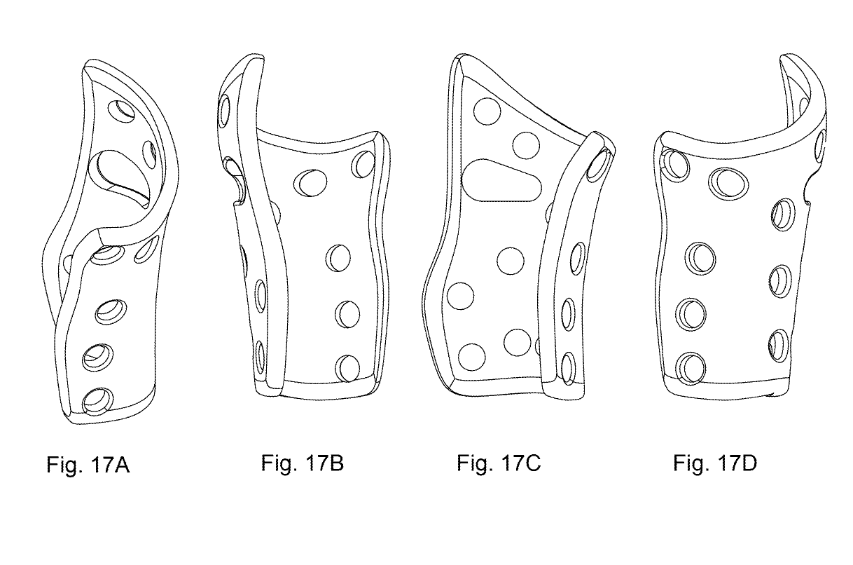

The following description is related to various aspects of olecranon fixation using the various implantable medical device embodiments described herein, and as shown in FIGS. 17 to 19.

As shown in FIG. 17, an olecranon plate can be made such that it is a contoured clamp, of various sizes, and orientations, e.g., right and left. In one embodiment, it has a viewing portal for viewing fractures during surgery and for viewing healing via an x-ray when implanted. As in other embodiments described herein, 3.5 mm screws variable locking and non-locking can be placed in unique and biomechanically secure configurations if screw fixation is desired. In one embodiment, a surgical instrument could be used to distract open the clamp, then released to tension around the fracture site. Besides being contoured to the bone they do not contact olecranon tip to avoid issue of irritating hardware. The method of using this type of clamp comprises exposing the fracture, reducing fracture, and applying the clamp/plate.

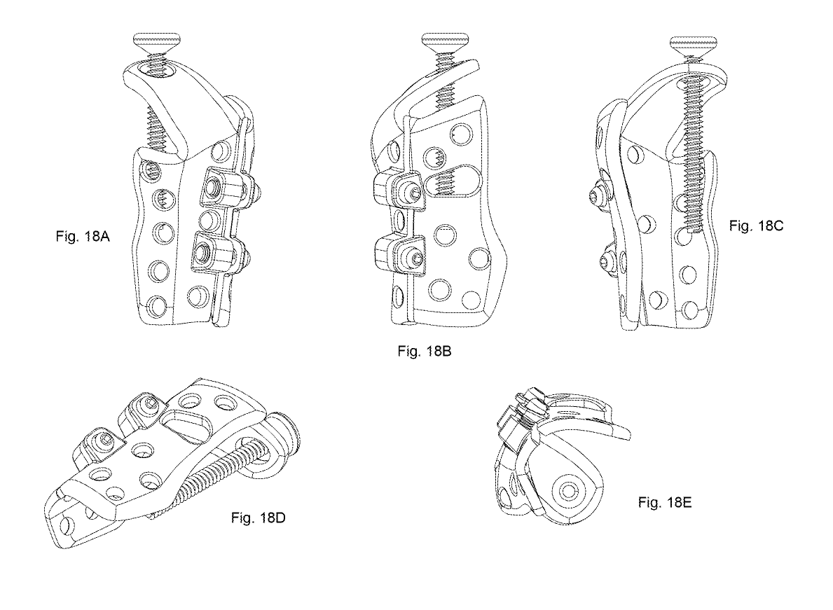

As shown in FIG. 18, in an another embodiment, there is disclosed an Olecranon plate that is separated into two pieces to allow it to more easily fit around bones of varying sizes. The two pieces are held to together by compression screws that apply compression around fracture site by tightening the screws. The design of this clamps allow for it to be applied to varying bone widths. While this plate is held together by the compression screw, it can be further stabilized by a rectangular peg and slot.

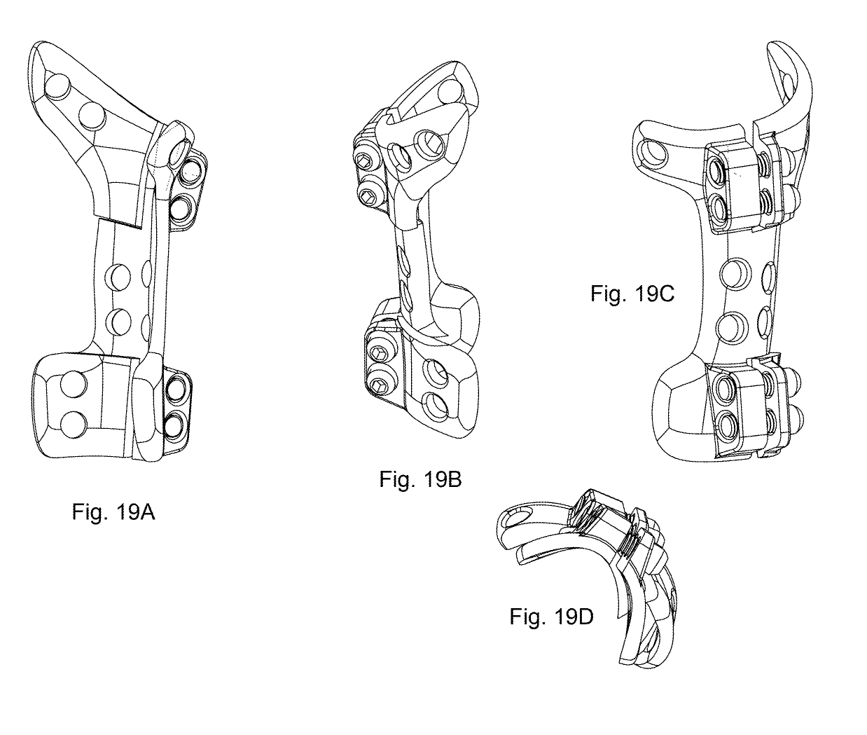

As shown in FIG. 19, in another embodiment, there is disclosed an Olecranon plate that takes the place of tension banding the Olecranon. It does curve around and contour to the tip of the Olecranon and has the ability to allow for intramedullary large screw (6.5 mm or 7.0 mm) fixation. The technique for applying this clamp comprises exposing the fracture, reducing it, applying the plate/clamp, then tightening the clamp. If necessary, an intramedullary supplemental screw can be used to enhance fixation.

In another embodiment, as shown in FIG. 19, there is disclosed an Olecranon plate that has two separate clamps, one to attach proximally to the Olecranon fractured end and one to attach to the ulna shaft. Again it is a low profile system, contoured, comes in various sizes, biomechanically more stable screw fixation if required.

The surgical technique differs, in that the proximal clamp would attach to the fractured end first, then the implant would reduce the fracture to the shaft and the distal and proximal clamp would be hand tightened to provide screwless fixation. Like the others described herein, this implant is used to perform the fracture reduction, and can incorporate a surgical instrument for holding the clamp and compress/tighten clamp as desired.

Humerus Plates

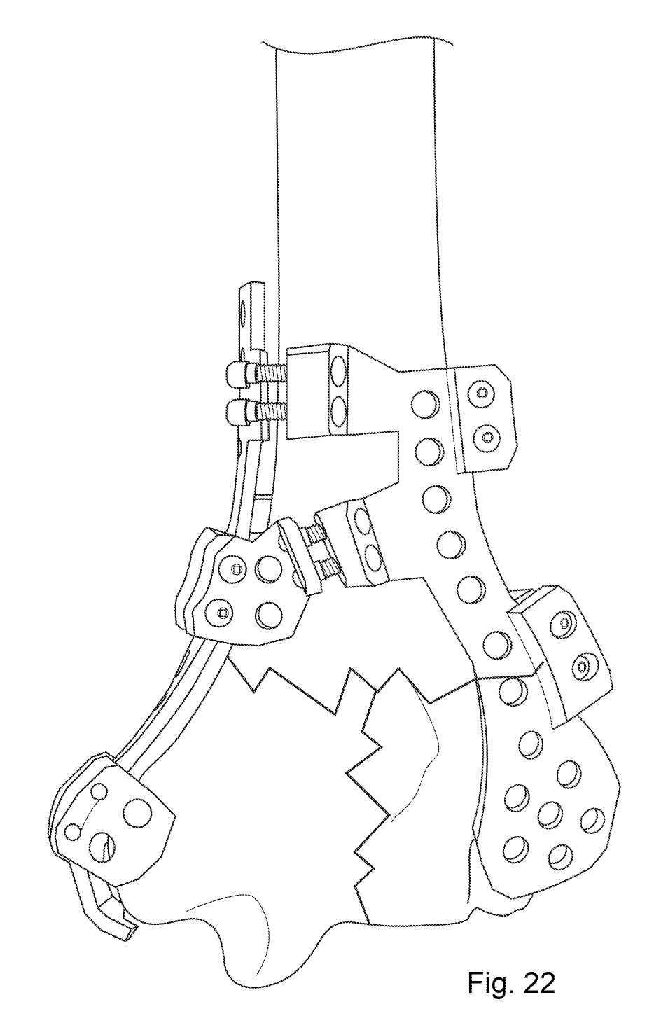

The following description is related to various aspects of humeral fixation using the various implantable medical device embodiments described herein, and as shown in FIGS. 22 to 24.

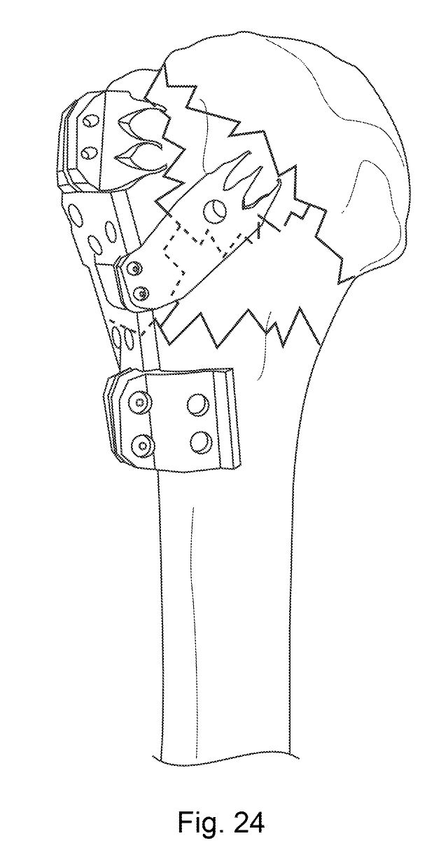

There is disclosed a method of attaching a proximal humeral plate to the greater tuberosity using an inventive clamp. In this embodiment, as shown in FIG. 24, the greater tuberosity and proximal Humeral head of the bone can be reduced to the shaft and then the clamp for the shaft can be applied to the Humeral shaft. The lesser tuberosity could then be clamped into position. If the case of humeral head mal-alignment, the inventive system comprises a hole for a pusher to maneuver it into position. Because both tuberosities have soft tissue attachments the clamps have teeth to grab the rotator cuff tendon and further secure the fracture sites.

In addition, the flexibility of the inventive device allows screws can be placed in unique geometries. For instance one could compress the lesser tuberosity to the shaft thru this screw placement. Various 4.5 mm, 3.5 mm locking, non-locking and variable angle screws can be used to secure fracture fixation. In one embodiment, the plates can be contoured to the shape of the bone, and made to be low profile. The clamps can then be positioned to secure the major fragments. The screw heads for the clamp are positioned in a manner to be easily accessible given surgical exposure. Holes closest to screw head are typically, but not always, non-threaded. One method of using this medical device can be varied to attach plate first to humerus than attach tuberosities if desired.

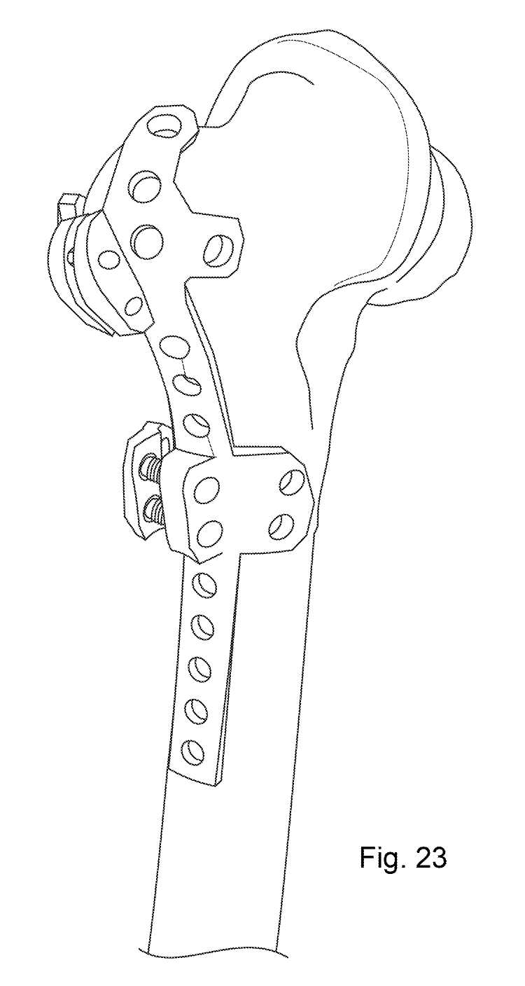

As shown in FIG. 22, in one embodiment there is disclosed a lateral condylar plate for the distal humerus, or a combined distal medial and lateral condylar plate. This implant is intended to be used for comminuted distal Humeral Fractures that need both medial and lateral columns supported. It can achieve screwless fixation of distal humerus fractures. The construct is contoured to fit around both medial and lateral columns. Screw fixation if desired can be placed in planes perpendicular to one another. In this embodiment, there are two clamps proximally that attach the two columnar plates to one another, they also serve to provide rapid and secure fixation to the Humeral shaft. Surgical technique for fixing such fractures can vary. In one embodiment, the distal articular surface can provisional be restored with a k-wire or a screw. In this method, the bone can be clamped distally and reduced to the Humeral shaft. The proximal clamps can then be tightened to provide proximal fixation. As shown in FIG. 23, the plates described herein can be combined in the distal humerus to increase functionality and fracture stability. An alternate technique could be to attach the plate construct proximally to the shaft than attach medial or lateral condyles to their respective clamps.

The description of the embodiments herein has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An implantable medical device for restoring bone alignment and stabilizing a fracture, comprising:
a screwless bone plating system for contacting at least a portion of a bone, the screwless bone plating system comprising:
at least one clamp comprising:
a top portion;
a bottom portion, and
an attachment for securing the clamp to a bone without drilling a screw into the bone,
wherein the at least one clamp is capable of realigning a fracture when applied to the bone, and remains implanted in the body to stabilize the fracture.

2. The implantable medical device of claim 1, wherein the attachment comprises one or more adjustable clamping members that engage the bone and secure the clamp to the bone with at least one fastener, the at least one fastener comprising at least one set screw, pin, peg, ratchet, or combination thereof.

3. The implantable medical device of claim 2, comprising two or more adjustable clamping members that have teeth to engage the bone and/or soft tissue surrounding the bone.

4. The implantable medical device of claim 2, wherein the at least one fastener is configured to maintain, adjust, or both maintain and adjust, the pressure that the one or more adjustable clamping members apply to the bone.

5. The implantable medical device of claim 1, wherein the bottom portion of the clamp comprises a snap-fit body that snaps onto a bone.

6. The implantable medical device of claim 5, wherein the snap-fit body comprises a metal or alloy that is in shape of the bone to be contacted that fits over a bone, and remains fixedly attached to the bone by compressive forces.

7. The implantable medical device of claim 1, wherein the clamp comprises a top portion comprising at least one hole or channel extending there-through, the hole or channel being adapted to receive a screw for attaching a bone plate to the clamp; and
a bottom portion comprising a metal or alloy containing body that snaps onto a bone, or that remains fixedly attached to a bone by compressive forces.

8. The implantable medical device of claim 7, further comprising at least one plate that is attached to at least one hole or channel in a fixed and unmovable position.

9. The implantable medical device of claim 7, further comprising at least one plate that is attached to the channel, which allows the plate to rotate with up to three degrees of freedom prior to the plate be fixedly secured to the clamp with one or more screws.

10. The implantable medical device of claim 7, comprising at least two of the clamps on opposite sides of a bone fracture, and at least one bone plate spanning the bone fracture and attached to the two clamps.

11. The implantable medical device of claim 1, wherein the top portion of the clamp comprises at least one hole or channel extending there-through, the hole or channel being adapted to receive a screw or connection post to attach the clamp to the bone, to a plate, to another clamp, or any combination thereof.

12. The implantable medical device of claim 11, further comprising at least one plate that is attached thereto, the plate comprising a body having at least one hole extending there-through, the hole being adapted to receive a screw or connection post to attach the plate to the bone, to another plate, to a clamp, or any combination thereof.

13. The implantable medical device of claim 12, wherein at least one plate is attached to the clamp in a fixed and unmovable position.

14. The implantable medical device of claim 12, wherein at least one plate is attached to the channel, which allows the plate to translate up and down the channel prior to the plate being fixedly secured to the clamp with one or more screws.

15. The implantable medical device of claim 14, wherein the top portion of the plate comprises a channel, and a screw connects the channel in the plate to the channel in the clamp such that the plate can translate and rotate with up to three degrees of freedom prior to the plate be fixedly secured to the clamp with one or more screws.

16. A modular fracture fixation system comprising two or more implantable medical devices of claim 1, wherein the two or more implantable medical devices comprise multiple clamps that can be positioned along the length of a bone, and proximal to a bone fracture.

17. The modular fracture fixation system of claim 16, further comprising at least one bone plate attached to one or more of the multiple clamps.

18. An implantable medical device for restoring bone alignment and stabilizing a fracture, comprising a screwless bone plating system for contacting at least a portion of a bone, the screwless bone plating system comprising:
at least two clamps for attaching to a bone, the clamps being located on opposite sides of a bone fracture and each comprising:
a top portion comprising at least one hole or channel extending there-through, the hole or channel being adapted to receive a screw or connection post for attaching a bone plate to the clamp; and
a bottom portion, wherein the bottom portion comprises an attachment for securing the clamp to a bone without drilling a screw into the bone;
at least one bone plate attached to the two clamps and spanning the bone fracture, wherein the at least two clamps are capable of realigning a fracture when applied to the bone, and remain implanted in the body to stabilize the fracture.

19. The implantable medical device of claim 18, wherein the bone plate is permanently attached to the clamps in a fixed and unmovable position.

20. The implantable medical device of claim 18, wherein the top portion of the plate comprises a channel, and a screw that connects the channel in the plate to the channel in the clamps such that the plate can translate and rotate with up to three degrees of freedom prior to the plate being fixedly secured to the clamps with one or more screws.

21. The implantable medical device of claim 18, wherein the top portion of the clamp comprises perpendicular channels for attaching a bone plate, and allowing the plate to translate along one of the channels.

22. The implantable medical device of claim 16, further comprising at least one bone plate positioned against the bone and underneath the clamp, wherein the clamp secures the bone plate to the bone.

23. The implantable medical device of claim 18, wherein the at least one bone plate comprises at least one channel running the length of the bone plate that allows at least one clamp to translate along the length of the bone plate.

24. The implantable medical device of claim 23, wherein at least one clamp comprise channels that run the width of the clamp, such that the clamp and the plate can translate in a perpendicular to each other.

25. A method of reducing and stabilizing a bone fracture, the method comprising:
contacting a fractured bone with an implantable medical device comprising:
a screwless bone plating system comprising:
at least one clamp comprising:
a top portion;
a bottom portion, and
an attachment for securing the clamp to a bone without drilling a screw into the bone;
attaching the at least one clamp to the fractured bone;
using the at least one clamp to help reduce the fracture by realigning the fractured ends of the bone;
translating the at least one clamp to the fracture site;
stabilizing the fracture by tightening the at least one clamp to the bone at the fracture site, wherein the clamp remains implanted as part of the permanent implant construct.

26. The method of claim 25, wherein the clamp is attached to the fractured bone with one or more adjustable clamping members that are locked into place by at least one fastener selected from a set screw, pin, peg, ratchet, or combinations thereof, wherein the at least one fastener is configured to maintain, adjust, or both maintain and adjust, the pressure that the adjustable clamping members apply to the bone.

27. The method of claim 25, wherein the clamp is attached to the fractured bone by snapping the bottom portion of the clamp to a fractured bone, the clamp remaining fixedly attached to the bone by compressive forces.

28. The method of claim 25, further comprising attaching at least one bone plate to the top portion of the clamp.

29. The method of claim 25, further comprising placing at least one bone plate directly on a bone, and securing the plate with the clamp.

30. The method of claim 27, wherein the clamp, the plate, or both the clamp and the plate comprise channels that run along their lengths and/or their widths, the method further comprising translating the plate along the channel to optimize the position of the plate prior to the plate be fixedly secured to the clamp.

31. The method of claim 30, wherein the clamp, the plate, or both the clamp are rotated up to three degrees of freedom prior to the plate be fixedly secured to the clamp.

32. The method of claim 25, further comprising attaching at least two of the clamps on opposite sides of a bone fracture, and attaching at least one bone plate to the two clamps such that the bone plate spans the bone fracture.

33. The method of claim 25, further comprising attaching multiple clamps along the length of a bone, and proximal to a bone fracture.

34. The method of claim 33, further comprising attaching at least one bone plate to one or more of the multiple clamps.

35. The method of claim 25, further comprising reducing multiple comminuted fractures by positioning at least three clamps to incrementally restore multiple fractures.

36. The method of claim 25, wherein the fractured bone is selected from clavicle, ulna, radius, carpal, humerus, phalanges, olecranon, and fibula.
